(19) United States
(12) Patent Application Publication
(10) Pub. No.: US 2025/0325720 A1
(43) Pub. Date: Oct. 23, 2025

(54) COMMUNICATION DEVICE, AND DISINFECTION AND STERILIZATION METHOD

(71) Applicant: ZHEJIANG GEELY HOLDING GROUP CO., LTD., Hangzhou (CN)

(72) Inventor: Shufu LI, Hangzhou (CN)

(73) Assignee: ZHEJIANG GEELY HOLDING GROUP CO., LTD., Hangzhou (CN)

(21) Appl. No.: 19/254,540

(22) Filed: Jun. 30, 2025

Related U.S. Application Data
(63) Continuation of application No. PCT/CN2023/123776, filed on Oct. 10, 2023.

(30) Foreign Application Priority Data
Dec. 30, 2022 (CN) 202211741615.0

(51) Int. Cl. A61L 2/10; A61L 2/24; H04M 1/72409

(52) U.S. Cl. CPC A61L 2/10; A61L 2/24; A61L 2202/11; A61L 2202/14; A61L 2202/16; H04M 1/72409

(57) ABSTRACT

Disclosed are a communication device and a disinfection and sterilization method. A terminal body is detachably and electrically connected to an ultraviolet sterilization apparatus or is in wireless communication connection with same, and via the terminal body, the ultraviolet sterilization apparatus is controlled to switch on to perform ultraviolet sterilization at a predetermined site, thereby ensuring that the ultraviolet sterilization apparatus can perform sterilization at a specific site within a small range at any time, which is simple and convenient; in addition, the ultraviolet sterilization apparatus can be completely separated from the terminal body when not in use, thereby preventing the terminal body from being excessively loaded due to the ultraviolet sterilization apparatus continuing to be arranged at the terminal body.

### CROSS-REFERENCE TO RELATED APPLICATIONS

This application is a continuation of International Application PCT/CN2023/123776, filed on Oct. 10, 2023, which claims priority to Chinese Patent Application No. 202211741615.0, filed on Dec. 30, 2022. All of the aforementioned applications are incorporated herein by reference in their entireties.

### TECHNICAL FIELD

The present disclosure relates to the technical field of communication devices, and more particularly, to a communication device, and a disinfection and sterilization method.

### BACKGROUND

With the development of smart phones, the functions of mobile phones are increasingly powerful. Currently, some mobile phones are provided with an ultraviolet lamp, and a control APP for the ultraviolet lamp is provided in the mobile phone. However, the current ultraviolet lamp in the mobile phone is generally used for checking bank notes, and the ultraviolet lamp of the bank note checking function is a false lamp, not a real ultraviolet lamp. With the development of society, new viral bacteria are emerging, which compactly appear in seats or computers in contact with people. If they are not killed in time, once the immunity of people is low, they will invade people and make them sick. Presently, sterilization for an enclosed area is performed by using UV lamp irradiation, which irradiates the entire area, requires complete sealing of the environment, and ensures that no personnel are present. As such, the use scenario is not applied to the personal area. The mobile phone is a necessary electronic product at present. When the ultraviolet lamp is set for irradiation, it is required to be considered that the usage frequency of the ultraviolet lamp is generally not high, and the load will be increased when the ultraviolet lamp is always set on the mobile phone.

### SUMMARY

An object of the first aspect of the present disclosure is to provide a communication device which solves the problems of the prior art in which an ultraviolet light source is provided directly at a terminal body, resulting in that the frequency of use thereof is too low and the load thereof is excessive.

Another object of the first aspect of the aspect of the present disclosure is to address the problem of ultraviolet light damage to humans.

An object of the second aspect of the disclosure is to provide a disinfection and sterilization method.

Specifically, the present disclosure provides a communication device, including a terminal body and an ultraviolet sterilization apparatus, wherein the terminal body is detachably and electrically connected to the ultraviolet sterilization apparatus, and the terminal body is used for providing electric energy for the ultraviolet sterilization apparatus; or

the terminal body is in wireless communication connection with the ultraviolet sterilization apparatus, and the terminal body is used for sending, to the ultraviolet sterilization apparatus, a control signal for switching on or switching off the ultraviolet sterilization apparatus.

Alternatively, the ultraviolet sterilization apparatus includes a housing and an ultraviolet light source provided at the housing, the housing being detachably connected to the terminal body.

Alternatively, the housing is detachably attached to a surface of the terminal body or detachably sleeved on a rear side of the terminal body.

Alternatively, the ultraviolet light source forms an image of a preset shape from at least one LED lamp bead, and the ultraviolet light source is located at a predetermined area range of the housing.

Alternatively, the preset shape is a strip shape or a surface shape.

Alternatively, it further includes a distance sensor configured for detecting a distance between the ultraviolet sterilization apparatus and a target object;
the terminal body further includes a sterilization control unit; and the sterilization control unit is connected to the distance sensor, and is configured for sending a prompt message prompting the user to approach or move away from the target object according to the detection result of the distance sensor.

Alternatively, the distance sensor is provided at the ultraviolet sterilization apparatus at a side of the ultraviolet light source.

Alternatively, the sterilization control unit is further configured for obtaining an effective sterilization distance of the target object according to the received intensity of the ultraviolet light wave emitted by the target object, determining whether the target object is within the effective sterilization distance according to the distance obtained by the distance sensor and the effective sterilization distance, and sending a prompt message prompting the user to approach or move away from the target object when the target object is not within the effective sterilization distance.

Alternatively, the sterilization control unit is further configured for calculating a distance between the target object and the ultraviolet light source based on a time of the ultraviolet wave emitted from the ultraviolet light source, a time of receiving the ultraviolet wave, and a wavelength of the ultraviolet wave after the distance sensor receives the ultraviolet wave emitted from the ultraviolet light source reflected from the target object.

Alternatively, the distance sensor includes a laser assembly, a receiving assembly and a processing module, wherein when the distance sensor is switched on, the laser assembly emits laser light; after the receiving assembly receives the light wave reflected by the target object, the distance between the target object and the ultraviolet light source is calculated by the processing module.

Alternatively, the terminal body further includes a display unit, wherein the display unit is connected to the sterilization control unit, and is configured for displaying the distance between the target object and the ultraviolet sterilization apparatus and the prompt message.

Alternatively, the terminal body further includes a rear camera for photographing an image at the rear of the terminal body; the rear camera is electrically connected to the sterilization control unit so as to transmit the photographed image information to the sterilization control unit; and the sterilization control unit then transmits the image information to the display unit and controls the display unit to display the image.

Alternatively, a first opening is provided at the housing; and
when the terminal body is engaged with the housing, the rear surface of the terminal body abuts against the housing, and the rear camera is located at the first opening.

Alternatively, the terminal body further includes a laser control unit electrically connected with the laser assembly, so that the laser control unit controls the terminal body to supply power to the laser light source or to switch on or off the laser light source when receiving a laser control instruction.

Alternatively, a laser light source is provided at the housing;
the terminal body further includes a laser control unit electrically connected with the laser light source, so that the laser control unit controls the terminal body to supply power to the laser light source or to switch on or off the laser light source when receiving a laser control instruction.

Alternatively, the laser light source is provided on a side of the ultraviolet light source.

Alternatively, a pressure sensor is provided at the left and/or right side of the housing or the terminal body; the pressure sensor is electrically connected to the sterilization control unit so as to transmit position information about a hand of the user holding the housing or the terminal body to the sterilization control unit after the pressure sensor monitors the position information; and the sterilization control unit determines, according to the position information, that the position of the hand of the user at least partially coincides with the predetermined area range, and controls whether to switch on the ultraviolet light source.

Alternatively, when the pressure sensor is provided at the terminal body, second openings are correspondingly provided on both sides of the housing, so that when the pressure sensor is fitted or sleeved together with the terminal body, the pressure sensor is exposed to the outside.

Specifically, the present disclosure also provides a sterilization and disinfection method applied to the communication device described above, the sterilization and disinfection method including:
when the terminal body is electrically connected to the ultraviolet sterilization apparatus, in response to a sterilization control instruction, controlling the terminal body to electrically supply power to the ultraviolet sterilization apparatus to switch on the ultraviolet sterilization apparatus, or controlling the terminal body to send a control signal to switch on or off the ultraviolet sterilization apparatus to switch on or off the ultraviolet sterilization apparatus.

Alternatively, after the ultraviolet sterilization apparatus is switched on, receiving a real-time distance between the ultraviolet sterilization apparatus and a target object obtained by a distance sensor, and the intensity of ultraviolet light reflected back by the target object;
obtaining an effective sterilization distance between the communication device and the target object according to the ultraviolet light intensity;
controlling the switching on of the ultraviolet light source when the real time distance is within the effective sterilization distance range; and
when the real-time distance is not within the effective sterilization distance range, controlling the ultraviolet light source to be switched off, and sending a prompt message prompting the user to change the implementation distance.

Alternatively, the method further includes:
obtaining position information about a hand of the user located at the communication device according to data obtained by a pressure sensor after the ultraviolet sterilization apparatus is switched on;
determining whether a position where the hand of the user is located at least partially coincides with a predetermined area range according to the position information, wherein the predetermined area range is an area range where an ultraviolet light source is provided at the communication device; and
when the position of the hand of the user at least partially coincides with the predetermined area range, controlling to send a warning message prompting the user of the improper hand position.

Alternatively, before controlling to switch on the ultraviolet light source, the method further includes:
determining whether a position where the hand of the user is located at least partially coincides with the predetermined area range; and
controlling the ultraviolet light source not to switch on until the hand of the user is not in the ultraviolet region irradiated by the ultraviolet light source when the position of the hand of the user at least partially coincides with the predetermined area range.

In this solution, a terminal body is detachably and electrically connected to an ultraviolet sterilization apparatus or is in wireless communication connection with same, and by means of the terminal body, the ultraviolet sterilization apparatus is controlled to switch on to perform ultraviolet sterilization at a predetermined site, thereby ensuring that the ultraviolet sterilization apparatus can perform sterilization at a specific site within a small range at any time, which is simple and convenient; in addition, the ultraviolet sterilization apparatus can be completely separated from the terminal body when not in use, thereby preventing the terminal body from being excessively loaded due to the ultraviolet sterilization apparatus continuing to be arranged at the terminal body.

Further, the ultraviolet light source of this aspect is a strip shape or a surface shape formed by at least one LED lamp bead. In general, the spot-shaped ultraviolet light source has limited sterilization effect. In this solution, the strip-shaped or planar light source is used, which can increase the area of light source, thus increasing the range and efficiency of sterilization and disinfection, and reducing the number and time of ultraviolet light source to achieve effective sterilization and disinfection while reducing the damage to human body.

Further, in the solution, a distance sensor is provided at the ultraviolet sterilization apparatus. The distance sensor can detect the distance between the ultraviolet sterilization apparatus and the target object and give a prompt according to the detected distance, so that the user can place the ultraviolet sterilization apparatus at a suitable position and distance from the target object, thereby ensuring that the ultraviolet sterilization apparatus is disinfected and sterilized within an effective range and improving the sterilization effect.

Further, the laser-emitting part in this aspect may be a separately provided laser light source provided at the housing and may be provided at a side of the ultraviolet light source. The laser light source is controlled by the laser control unit so that the laser control unit can control the terminal body to supply power to the laser light source while controlling the laser light source to be switched on or off. This allows the terminal body to function as a laser pen in addition to a communication device.

In this solution, a pressure sensor is provided on both sides of the terminal body, and the pressure sensor can monitor the position of the finger of the user when in use so as to transmit to the sterilization control unit. Then, it is estimated whether the position of the finger overlaps with the position of the ultraviolet light source. If it is overlapped, the ultraviolet light source cannot be switched on at this time, so as to prevent ultraviolet light from directly irradiating on the hand of the user, so as to avoid ultraviolet radiation damage to the user.

In this solution, the ultraviolet sterilization apparatus which can be electrically connected with the terminal body is used, and the ultraviolet sterilization apparatus is powered or controlled to perform disinfection and sterilization on the target object by the terminal body, so that the terminal body can be transformed into a sterilization device, and the terminal body carried by the user can perform disinfection and sterilization on the peripheral range of the user at any time, thus reducing the damage to the human body caused by bacteria or viruses. In addition, the detachable arrangement or detachable electrical connection between the ultraviolet sterilization apparatus and the terminal body in the present solution can be closed or even directly separated from the terminal body when the ultraviolet sterilization apparatus is not in use, without causing excessive load on the terminal body.

The above and other objects, advantages and features of the present disclosure will become more apparent to those skilled in the art from the following detailed description of specific embodiments of the disclosure when taken in conjunction with the accompanying drawings.

### BRIEF DESCRIPTION OF THE DRAWINGS

Specific embodiments of the disclosure will be described in detail hereinafter, by way of example and not limitation, with reference to the accompanying drawings. The same reference numbers will be used throughout the drawings to refer to the same or like parts or parts. Those skilled in the art should appreciate that the figures are not necessarily drawn to scale. In the drawings:

FIG. 1 is a schematic block diagram of a communication device according to a specific embodiment of the present disclosure;

FIG. 2 is a connection block diagram of a communication device according to a specific embodiment of the present disclosure;

FIG. 3 is a connection block diagram of a communication device according to another specific embodiment of the present disclosure;

FIG. 4 is a connection block diagram of a communication device according to yet another specific embodiment of the present disclosure;

FIG. 5 is a connection block diagram of a communication device according to yet another specific embodiment of the present disclosure;

FIG. 6 is a schematic flow diagram of a disinfection and sterilization method according to a specific embodiment of the present disclosure;

FIG. 7 is a schematic flow diagram of a disinfection and sterilization method according to another specific embodiment of the present disclosure;

FIG. 8 is a schematic flow diagram of a disinfection and sterilization method according to yet another specific embodiment of the present disclosure; and

FIG. 9 is a schematic flow chart of a disinfection and sterilization method according to yet another specific embodiment of the present disclosure.

### DETAILED DESCRIPTION OF THE EMBODIMENTS

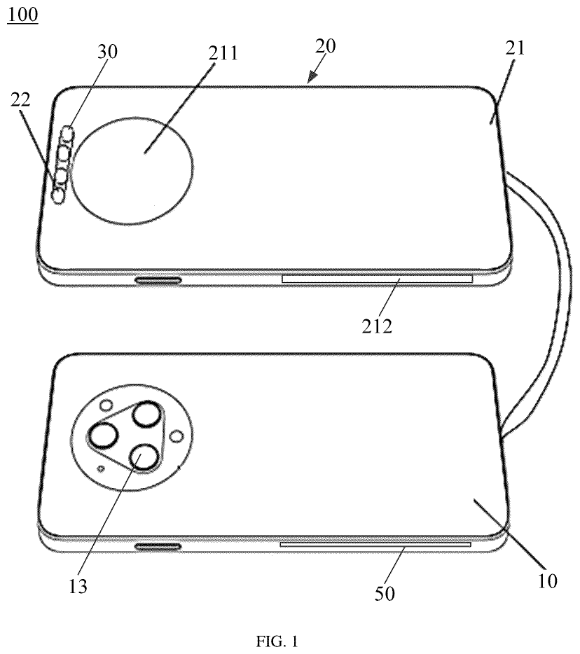

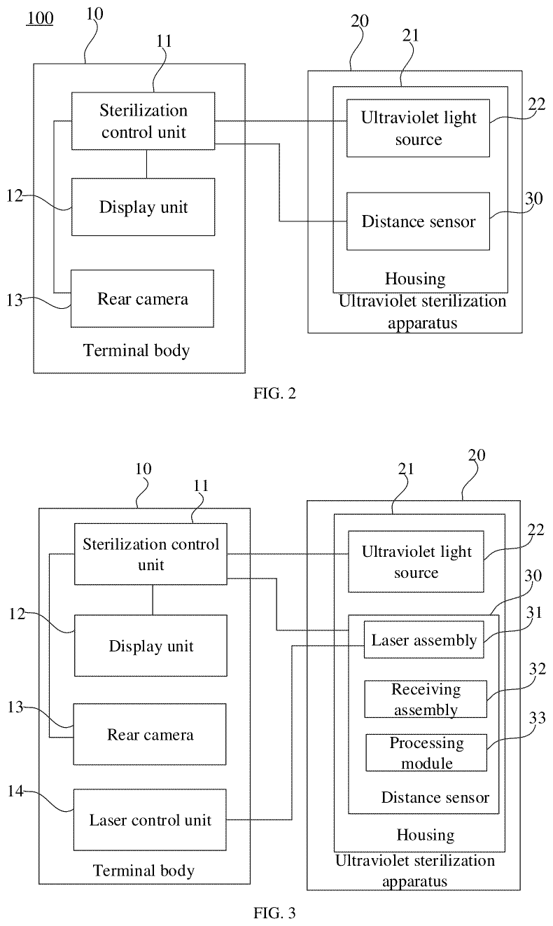

FIG. 1 is a schematic block diagram of a communication device according to a specific embodiment of the present disclosure. FIG. 2 is a connection block diagram of a communication device according to a specific embodiment of the present disclosure. As a specific embodiment of the present disclosure, the present embodiment provides a communication device 100 which may include a terminal body 10 and an ultraviolet sterilization apparatus 20. The terminal body 10 is detachably electrically connected to the ultraviolet sterilization apparatus 20 for supplying power to the ultraviolet sterilization apparatus 20. The terminal body 10 is in wireless communication connection with the ultraviolet sterilization apparatus 20. The terminal body 10 is used for sending, to the ultraviolet sterilization apparatus 20, a control signal for switching on or switching off the ultraviolet sterilization apparatus 20.

Specifically, in the present embodiment, the terminal body 10 and the ultraviolet sterilization apparatus 20 are detachably electrically connected to each other in a pluggable manner. In other words, there is a pluggable structure between the ultraviolet sterilization apparatus 20 and the terminal body 10. When the ultraviolet sterilization apparatus 20 is inserted into the terminal body 10 by the pluggable structure, the terminal body 10 can supply electric power to the ultraviolet sterilization apparatus 20, and the ultraviolet sterilization apparatus 20 can be used. In this case, the terminal body 10 can not only supply power to the ultraviolet sterilization apparatus 20 but also control the use of the ultraviolet sterilization apparatus 20. However, when the ultraviolet sterilization apparatus 20 is separated from the terminal body 10, the ultraviolet sterilization apparatus 20 has no power source and cannot be used.

As another embodiment, in the present embodiment, the terminal body 10 is connected to the ultraviolet sterilization apparatus 20 in an unlimited communication. The ultraviolet sterilization apparatus 20 and the terminal body 10 may be mechanically fastened to each other or separated from each other by an unlimited communication. Since the ultraviolet sterilization apparatus 20 and the terminal body 10 are connected in wireless communication. The ultraviolet sterilization apparatus 20 needs a power supply for normal use. A power supply, a communication module communicatively connected to the terminal body 10 and an execution module for executing an action are required to be added in the ultraviolet sterilization apparatus 20 in the present embodiment. When the terminal body 10 sends a control signal for switching on or switching off the ultraviolet sterilization apparatus 20 to the ultraviolet sterilization apparatus 20, the communication module receives the signal and performs an action using the execution module. Of course, it is preferable that a control module, which communicates with the terminal body 10 and controls the ultraviolet sterilization apparatus 20 to perform a corresponding action, be provided in the ultraviolet sterilization apparatus 20.

Specifically, in the present embodiment, a terminal body 10 is detachably and electrically connected to an ultraviolet sterilization apparatus 20 or is in wireless communication connection with same, and by means of the terminal body 10, the ultraviolet sterilization apparatus 20 is controlled to switch on to perform ultraviolet sterilization at a predetermined site, thereby ensuring that the ultraviolet sterilization apparatus 20 can perform sterilization at a specific site within a small range at any time, which is simple and convenient. In addition, the ultraviolet sterilization apparatus 20 can be completely separated from the terminal body 10 when not in use, thereby preventing the terminal body 10 from being excessively loaded due to the ultraviolet sterilization apparatus 20 continuing to be arranged at the terminal body 10.

As a specific embodiment of the present disclosure, the ultraviolet sterilization apparatus 20 of the present embodiment may include a housing 21 and an ultraviolet light source 22 provided at the housing 21, the housing 21 being detachably connected to the terminal body 10.

Specifically, the ultraviolet sterilization apparatus 20 of the present embodiment is provided with a housing 21 and an ultraviolet light source 22 located at the housing 21. The housing 21 may be detachably connected to the terminal body 10. The housing 21 may be of any shape and size. Of course, it is preferable that if the terminal body 10 is a mobile phone or a tablet computer. The housing 21 may be formed in the form of a mobile phone housing or a tablet computer housing 21. The housing 21 is directly sleeved on the rear side of the terminal body 10 when used by a user. Of course, as other embodiments, the housing 21 may be formed in a sheet shape having a specific shape and adhered to the outer surface of the terminal body 10 using an adhesive. For example, in an embodiment, if the terminal body 10 is a mobile phone, the housing 21 may be a circular or square or other shaped sheet structure. The housing 21 is adhered to the back surface of the mobile phone, or the side surface of the mobile phone, or the upper end of the mobile phone using an adhesive. In addition, if the terminal body 10 and the ultraviolet sterilization apparatus 20 are detachably electrically connected, the housing 21 may be formed in a U-disk-like structure to be inserted at a specific position of the mobile phone.

Of course, regardless of the structure of the housing 21, the ultraviolet light source 22 is required to be exposed to the outside when the housing 21 and the terminal body 10 are connected to each other to ensure that ultraviolet rays can be emitted to sterilize the external environment or articles.

As a specific embodiment of the present disclosure, the ultraviolet light source 22 of the present embodiment forms an image of a preset shape from at least one LED lamp bead, and the ultraviolet light source 22 is located at a predetermined area range of the housing 21.

Specifically, the ultraviolet light source 22 is a strip shape or a surface shape formed by at least one LED lamp bead. In general, the spot-shaped ultraviolet light source 22 has limited sterilization effect. In this solution, the adjusted or planar light source is used, which can increase the area of light source, thus increasing the range and efficiency of sterilization and disinfection, and reducing the number and time of ultraviolet light source 22 to achieve effective sterilization and disinfection while reducing the damage to human body.

Preferably, the ultraviolet light source 22 in the present embodiment may occupy ½ of the area of the terminal body 10 when the ultraviolet sterilization apparatus 20 is fitted or fastened together with the terminal body 10, and be located at the upper portion. The remaining position is left for the user to hold the terminal body 10.

Specifically, the predetermined area in the present embodiment can also be set as needed. Generally, the predetermined area can be the same as the shape of the image of the ultraviolet light source 22, but the area will be larger. The ultraviolet light source 22, when shining outwards, is actually a divergent light source, the farther away from the ultraviolet light source 22, the larger the area of its light spot.

As a specific embodiment of the present disclosure, the communication device 100 of the present embodiment may further include a distance sensor 30 configured for detecting a distance between the ultraviolet sterilization apparatus 20 and a target object. The terminal body 10 may further include a sterilization control unit 11 connected to the distance sensor 30 for emitting a prompt message prompting the user to approach or move away from the target object according to the detection result of the distance sensor 30.

Specifically, in practical applications, since the distance between the ultraviolet light source 22 and the target object affects the sterilization effect, in general, the longer the distance, the worse the sterilization effect. Specifically, since the ultraviolet sterilization apparatus 20 in the present embodiment is a device which can be integrated on a mobile phone, the device is small, and the sterilization effect is not optimistic for a target object having a relatively long distance. The present embodiment provides a distance sensor 30 at the ultraviolet sterilization apparatus 20. The distance sensor 30 can detect the distance between the ultraviolet sterilization apparatus 20 and the target object and give a prompt according to the detected distance, so that the user can place the ultraviolet sterilization apparatus 20 at a suitable position and distance from the target object, thereby ensuring that the ultraviolet sterilization apparatus 20 is disinfected and sterilized within an effective range and improving the sterilization effect.

As a specific embodiment of the present disclosure, the distance sensor 30 of the present embodiment is provided at the ultraviolet sterilization apparatus 20, and the distance sensor 30 is provided at a side of the ultraviolet light source 22.

Specifically, since the distance sensor 30 specifically needs to know the distance between the ultraviolet light source 22 and the target object, the distance sensor 30 is disposed at the ultraviolet sterilization apparatus 20. If the distance sensor 30 is provided at the terminal body 10, the ultraviolet sterilization apparatus 20 is required to be engaged with the terminal body 10 to detect the distance between the ultraviolet light source 22 and the target object using the distance sensor 30.

In the present embodiment, the distance sensor 30 is directly provided at the ultraviolet sterilization apparatus 20, and is located at a side of the ultraviolet light source 22, so that it can be ensured that the distance sensor 30 detects the distance between the ultraviolet light source 22 and the target object. It is not necessary to attach or fasten the ultraviolet sterilization apparatus 20 and the terminal body 10 to each other, thereby improving the convenience of use.

As a specific embodiment of the present disclosure, the sterilization control unit 11 of the present embodiment may be further configured for obtaining an effective sterilization distance of the target object according to the received intensity of the ultraviolet light wave emitted by the target object, determining whether the target object is within the effective sterilization distance according to the distance obtained by the distance sensor 30 and the effective sterilization distance, and sending a prompt message prompting the user to approach or move away from the target object when the target object is not within the effective sterilization distance.

Specifically, in the present embodiment, by receiving the intensity of the ultraviolet wave reflected by the target object, it is possible to know the intensity of the ultraviolet wave irradiated on the target object by the ultraviolet wave, and the ultraviolet wave having a sterilization function generally needs to reach a certain intensity. Therefore, if the intensity of the received ultraviolet wave is within the intensity range, it means that the intensity of the ultraviolet wave irradiated on the target object by the ultraviolet light source 22 has a sterilization function, and the distance is feasible. If the intensity of the ultraviolet wave reflected by the target object received is weak, the sterilization effect may not be good at this time, and it is necessary to appropriately move the distance between the ultraviolet light source 22 and the target object. At this time, a prompt sound may be given to the user.

As a specific embodiment of the present disclosure, the sterilization control unit 11 of the present embodiment is further configured for calculating a distance between the target object and the ultraviolet light source 22 based on the time of the ultraviolet wave emitted from the ultraviolet light source 22, the time of receiving the ultraviolet wave, and the wavelength of the ultraviolet wave after the distance sensor 30 receives the ultraviolet wave emitted from the ultraviolet light source 22 reflected from the target object.

Specifically, the distance between the ultraviolet light source 22 and the target object is determined depending on the time when the light wave is emitted, the time when the light wave is reflected, and the wavelength of the light wave. Thus, the distance between the target object and the ultraviolet light source 22 can be calculated by the time when the light wave is emitted from the ultraviolet light source 22 and the time and wavelength when the light wave reflected from the target object by the light wave emitted from the ultraviolet light source 22 is received. At this time, the distance sensor 30 is mainly used for receiving the reflected ultraviolet light wave and calculating the distance.

FIG. 3 is a connection block diagram of a communication device according to another specific embodiment of the present disclosure. As another specific embodiment of the present disclosure, the distance sensor 30 of the present embodiment may include a laser assembly 31, a receiving assembly 32 and a processing module 33. When the distance sensor 30 is switched on, the laser assembly 31 emits laser light. After the receiving assembly 32 receives the light wave reflected by the target object, the distance between the target object and the ultraviolet light source 22 is calculated by the processing module 33.

In the present embodiment, the distance sensor 30 itself has a laser assembly 31, a receiving assembly 32, and a processing module 33. The laser assembly 31 emits laser light, which is partially reflected back to be received by the receiving assembly 32 after being irradiated on the surface of the target object. The distance sensor 30 can record the time of emitting laser light and the time of receiving laser light, and then calculate the distance between the distance sensor 30 and the target object together with the wavelength of the laser light. In general, the distance sensor 30 is provided on a side of the ultraviolet light source 22 of the ultraviolet sterilization apparatus 20 such that the calculated distance can be considered to be the distance between the ultraviolet sterilization apparatus 20 and the target object.

As a specific embodiment of the present disclosure, the terminal body 10 of the present embodiment may further include a display unit 12 connected to the sterilization control unit 11 for displaying a distance between the target object and the ultraviolet sterilization apparatus 20 and a prompt message.

Specifically, in general, in the present application, when the distance sensor 30 is switched on, the display unit 12 is also switched on when the distance sensor 30 is switched on, so that the distance information detected by the distance sensor 30 can be displayed at the display unit 12. The user can more intuitively sense the distance between the ultraviolet light source 22 and the target object.

Of course, as a specific embodiment of the present disclosure, the terminal body 10 of the present embodiment may further include a rear camera 13 for photographing an image at the rear of the terminal body 10. The rear camera 13 is electrically connected to the sterilization control unit 11 to transmit the photographed image information to the sterilization control unit 11. The sterilization control unit 11 transmits the image information to the display unit 12 and controls the display unit 12 to display the image.

In the above-mentioned embodiments, if the ultraviolet sterilization mode is switched on in the form of a mobile phone APP in the present application, both the display unit 12 and the rear camera 13 located at the terminal body 10 are synchronously switched on while being switched on. The distance sensor 30 firstly needs to detect the distance between the ultraviolet light source 22 and the target object. The rear camera 13 takes an image of the target object, and then synchronously displays the image and the distance information at the display unit 12, so that a user can intuitively know the area of the target object to be sterilized and disinfected.

Specifically, as described above, since it is necessary to know whether the distance between the target object and the ultraviolet light source 22 is a suitable distance, when the ultraviolet sterilization mode is switched on, the ultraviolet light source 22 will also emit a segment of ultraviolet light for a short time and analyze the intensity of the ultraviolet light reflected by the ultraviolet light source 22 after being received, so as to obtain whether the distance is within a suitable distance range. If the distance is within the suitable range, the ultraviolet light source 22 will be further switched on. If not, the user will be prompted to change the distance between the ultraviolet light source 22 and the target object until the ultraviolet light source 22 will continuously emit ultraviolet light when the distance reaches the effective sterilization distance.

As a specific embodiment of the present disclosure, a first opening 211 is provided at the housing 21 of the present embodiment. When the terminal body 10 is engaged with the housing 21, the rear surface of the terminal body 10 abuts against the housing 21, and the rear camera 13 is located at the first opening 211. Specifically, since the ultraviolet sterilization apparatus 20 in the present application may be provided in the form of a housing 21, the housing 21 may be a mobile phone case provided at the rear of the mobile phone. When the mobile phone shell is in use, the mobile phone shell may be sleeved behind the mobile phone, and a rear camera 13 is provided behind the mobile phone, and the camera needs to pass through the mobile phone shell to capture an item behind the mobile phone shell. Therefore, a first opening 211 is required to be provided at the mobile phone shell, and the rear camera 13 is provided at the first opening 211, so as to ensure that the rear camera 13 can pass through the first opening 211 to capture image information about a target object behind the mobile phone shell.

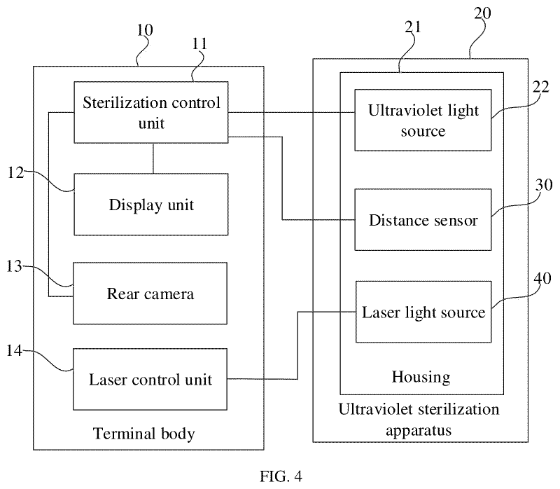

FIG. 4 is a connection block diagram of a communication device according to yet another specific embodiment of the present disclosure. As a specific embodiment of the present disclosure, the terminal body 10 of the present embodiment may further include a laser control unit 14 electrically connected to the laser assembly 31 such that the laser control unit 14 controls the terminal body 10 to supply power to the laser light source or to switch on or off the laser light source when receiving a laser control instruction.

Specifically, in the present embodiment, since the distance sensor 30 includes the laser unit 31, it can emit laser light with which the terminal body 10 can be used as a laser pen, thereby increasing the application range of the terminal body 10. The distance sensor 30 can be used not only to switch on laser light emitting at a predetermined wavelength according to need when ultraviolet sterilization is performed, but also to emit laser light when it is required to be converted into a laser pen.

Specifically, the laser assembly 31 may be controlled by the sterilization control unit 11 together with the sterilization control unit 11 as an assembly for distance measurement. In addition, the laser assembly 31 may be connected to the laser control unit 14 to emit laser light under the control of the laser control unit 14.

Specifically, the laser control unit 14 of the present embodiment may be provided on the terminal body 10, which controls the laser assembly 31 to emit laser light in response to an operation instruction when the APP on the terminal body 10 is operated.

As a specific embodiment of the present disclosure, the housing 21 of this embodiment is provided with a laser light source 40. The terminal body 10 further includes a laser control unit 14 electrically connected to the laser light source 40 such that the laser control unit 14 controls the terminal body 10 to supply power to the laser light source 40 or to switch on or off the laser light source 40 when receiving a laser control instruction.

In the present embodiment, the laser-emitting component may be a separately provided laser light source 40 provided at the housing 21 and may be provided on a side of the ultraviolet light source 22. The laser light source 40 is controlled by the laser control unit 14 so that the laser control unit 14 can control the terminal body 10 to supply power to the laser light source 40 while controlling the laser light source 40 to be switched on or off. This allows the terminal body 10 to function as a laser pen in addition to a communication device 100.

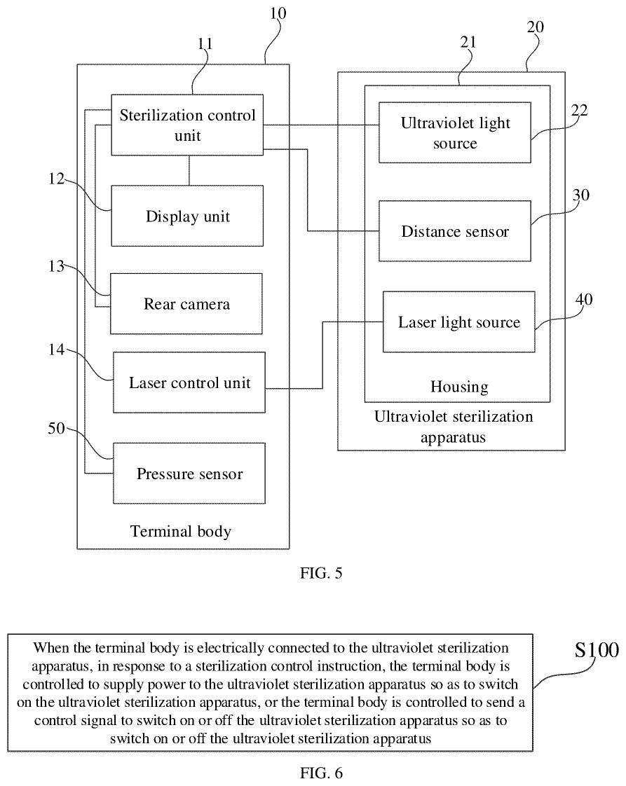

FIG. 5 is a connection block diagram of a communication device according to yet another specific embodiment of the present disclosure. As a specific embodiment of the present disclosure, a pressure sensor 50 is provided at the left and/or right side of the housing 21 or the terminal body 10 in the present embodiment. The pressure sensor 50 is electrically connected to the sterilization control unit 11 so as to transmit position information to the sterilization control unit 11 after the pressure sensor 50 monitors the position information about the hand of the user holding the housing 21 or the terminal body 10. The sterilization control unit 11 determines that the position of the hand of the user at least partially coincides with the predetermined area range according to the position information, and controls whether to switch on the ultraviolet light source.

Specifically, in general, the ultraviolet light is harmful to the human body. When the ultraviolet light is irradiated on the hand of the user or other parts of the body for a long time, the irradiated parts are easily damaged, blackened in a mild case and being cancer in a severe case. Therefore, in order to avoid this, the present embodiment is provided with a pressure sensor 50 on both sides of the terminal body 10, and the pressure sensor 50 can monitor the position of the finger of the user when in use, thereby transmitting to the sterilization control unit 11. Since the position of the ultraviolet light source 22 is relatively fixed when the ultraviolet light source 22 is designed, it is possible to estimate whether the position of the finger overlaps with the position of the ultraviolet light source 22 when the position of the finger is obtained. If it is overlapped, the ultraviolet light source 22 cannot be switched on at this time to prevent the ultraviolet light from being directly irradiated on the hand of the user.

Of course, the pressure sensor 50 is used in a case where the housing 21 and the terminal body 10 are fitted with each other, if the housing 21 and the terminal body 10 are not fitted with each other, since the ultraviolet light source 22 is provided at the housing 21 and the user holds the terminal body 10 at this time, it is irrelevant whether the ultraviolet light irradiated from the ultraviolet light source 22 is recombined with the finger. At this time, it is possible to use the picture taken by the rear camera 13 to determine whether there is a person in the ultraviolet light irradiated area or the body part of the user, and thus it is possible to actively choose to replace or remind the user of the need to quickly transfer the position so as to avoid injury.

As a specific embodiment of the present disclosure, when the pressure sensor 50 of the present embodiment is provided at the terminal body 10, the second openings 212 are correspondingly provided at both sides of the housing 21 so that the pressure sensor 50 is exposed when fitted or sleeved with the terminal body 10.

Specifically, in the present embodiment, the pressure sensor 50 is provided at the terminal body 10, which is required to be exposed and can be pressed by the hand of the user when the housing 21 is sleeved with the terminal body 10, so that the second opening is provided at the housing 21. Thus, the pressure sensor 50 can be exposed and pressed by the hand of the user when the housing 21 is sleeved with the terminal body 10.

Specifically, in the present embodiment, the terminal body 10 can be a mobile phone, a pad, etc. In the present embodiment, it is preferred that the terminal body 10 is a mobile phone. The ultraviolet sterilization apparatus 20 is provided at the mobile phone housing. The mobile phone housing and the mobile phone can be fastened to each other or can be disassembled and placed separately. The connection between the mobile phone shell and the terminal body 10 may be a wired connection or a wireless connection. A sterilization control unit 11 is provided in the terminal body 10, and supplies power to the ultraviolet light source 22 or the controller is switched on or off in response to an operation instruction from the outside. In addition, a distance sensor 30 is provided at the mobile phone shell, so that the distance between the housing 21 and the target object can be detected, and the effective sterilization distance of the ultraviolet light source 22 from the target object can also be obtained by using the light intensity of the reflected light at the target object irradiated by the ultraviolet light emitted by the ultraviolet light source 22, thereby ensuring the effectiveness of sterilization. In addition, in the present application, a rear camera 13 and a display unit 12 are provided at the mobile phone. When the sterilization control unit 11 turns on in response to an external instruction, the rear camera 13 and the display unit 12 of the mobile phone are controlled to switch on at the same time, a picture taken by the rear camera 13 is displayed at the display unit 12, and both the distance detected by the distance sensor 30 and the effective sterilization distance are displayed at the display unit 12. The user can more intuitively see the sterilization range of the ultraviolet sterilization apparatus 20 and the distance from the target object, so that the sterilization effect can be better ensured.

In addition, the present embodiment is further provided with a laser light source 40 at the mobile phone housing 21, and a laser control unit 14 is provided in the mobile phone. The laser control unit 14 can also supply power to the laser light source 40 or control the laser light source 40 to be switched on or off in response to a control instruction.

In addition, in the present embodiment, a pressure sensor 50 can be provided at the left and right sides of the mobile phone, can detect the position of a finger in real time when the ultraviolet sterilization apparatus 20 is switched on, and display the same at the display unit 12. The ultraviolet light source 22 is switched on only when the position of the finger does not coincide with the position of the ultraviolet light emitted from the ultraviolet light source 22, thus effectively protecting the user.

FIG. 6 is a schematic flow diagram of a disinfection and sterilization method according to a specific embodiment of the present disclosure. As a specific embodiment of the present disclosure, the present embodiment also provides a sterilization and disinfection method applied to the above communication device 100. The sterilization and disinfection method may include the step below.

Step S100, when the terminal body 10 is electrically connected to the ultraviolet sterilization apparatus 20, in response to a sterilization control instruction, the terminal body 10 is controlled to electrically supply power to the ultraviolet sterilization apparatus 20 to switch on the ultraviolet sterilization apparatus 20, or the terminal body 10 is controlled to send a control signal to switch on or off the ultraviolet sterilization apparatus 20 to switch on or off the ultraviolet sterilization apparatus 20.

In the present embodiment, the ultraviolet sterilization apparatus 20 can be electrically connected with the terminal body 10, the ultraviolet sterilization apparatus 20 can be powered or controlled by the terminal body 10 to perform disinfection and sterilization on the target object, so that the terminal body 10 can be converted into a sterilization device, and the terminal body 10 carried by the user can perform disinfection and sterilization on the peripheral range of the user at any time, reducing the damage to the human body caused by bacteria or viruses. In addition, the detachable arrangement or detachable electrical connection between the ultraviolet sterilization apparatus 20 and the terminal body 10 of the present embodiment can be closed or even directly separated from the terminal body 10 when the ultraviolet sterilization apparatus 20 is not in use, without causing excessive load on the terminal body.

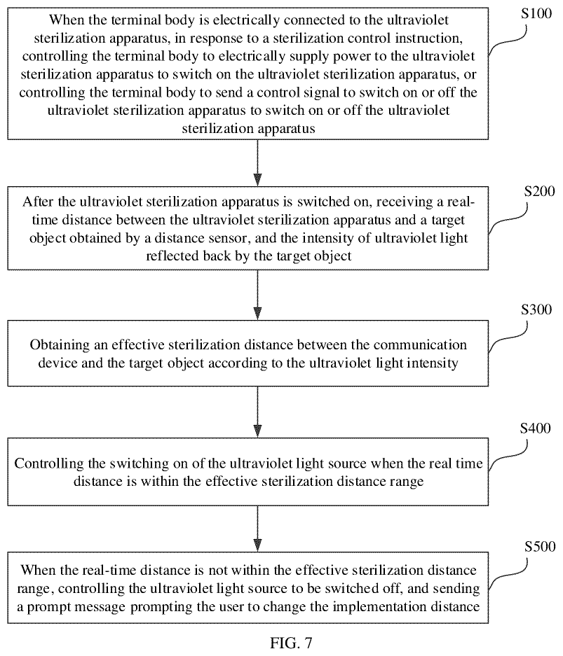

FIG. 7 is a schematic flow diagram of a disinfection and sterilization method according to another specific embodiment of the present disclosure. As a specific embodiment of the present disclosure, the disinfection and sterilization method of the present embodiment may further include the steps below:
step S200, after the ultraviolet sterilization apparatus 20 is switched on, a real-time distance between the ultraviolet sterilization apparatus 20 and a target object obtained by a distance sensor 30, and the intensity of ultraviolet light reflected back by the target object are received;
step S300, an effective sterilization distance is obtained between the communication device 100 and the target object according to the ultraviolet light intensity;
step S400, the switching on of the ultraviolet light source 22 is controlled when the real-time distance is within the effective sterilization distance range; and
step S500, when the real-time distance is not within the effective sterilization distance, the ultraviolet light source 22 is controlled to be switched off, and a prompt message prompting the user to change the implementation distance is sent.

In the present embodiment, the effective sterilization distance of ultraviolet rays is calculated before the ultraviolet light source 22 of the ultraviolet sterilization apparatus 20 is switched on, and then the distance between the ultraviolet light source 22 and the target object at that moment is detected. The ultraviolet light source 22 is switched on or off by the distance control, so as to ensure the sterilization effect of the ultraviolet device.

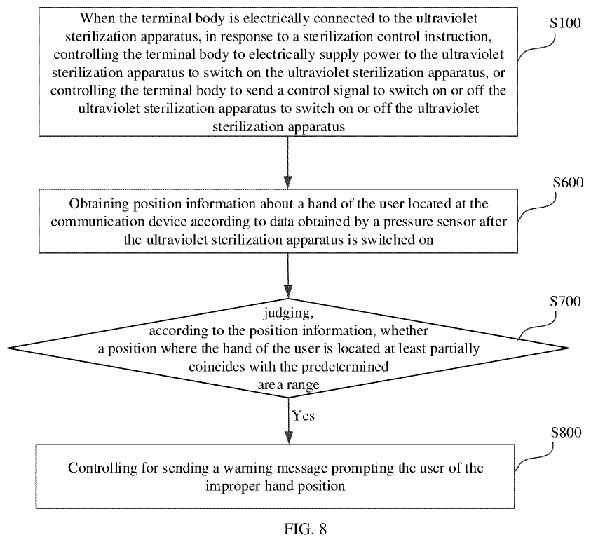

FIG. 8 is a schematic flow chart of a disinfection and sterilization method according to yet another specific embodiment of the present disclosure. As a specific embodiment of the present disclosure, the disinfection and sterilization method of the present embodiment may further include the steps below:
step S600, position information about a hand of the user located at the communication device 100 is obtained according to the data obtained by the pressure sensor 50 after the ultraviolet sterilization apparatus 20 is switched on;
step S700, it is determined whether a position where the hand of the user is located at least partially coincides with a predetermined area range according to the position information, wherein the predetermined area range is an area range where an ultraviolet light source 22 is provided at the communication device 100; and
step S800, when the position of the hand of the user at least partially coincides with the predetermined area range, it is controlled to send a warning message prompting the user of the improper hand position.

In the present embodiment, the position of the hand is detected before the ultraviolet light source 22 is switched on, and then it is obtained whether the ultraviolet light emitted by the ultraviolet light source 22 will irradiate the hand. When the ultraviolet light can irradiate the hand, it is necessary to prompt the user to remove the hand in time so as to avoid the injury to the hand caused by the ultraviolet light. In the present embodiment, the pressure sensor 50 is used to obtain the position information of the hand of the user, and the position of the ultraviolet light source 22 and the angle information of the emitted light can be used to obtain whether the ultraviolet light source 22 will irradiate the hand.

Of course, as another embodiment, it is also possible to directly determine whether the hand position blocks the ultraviolet light by the camera irradiation, so that it is possible to more intuitively prevent the hand from being damaged by the ultraviolet irradiation.

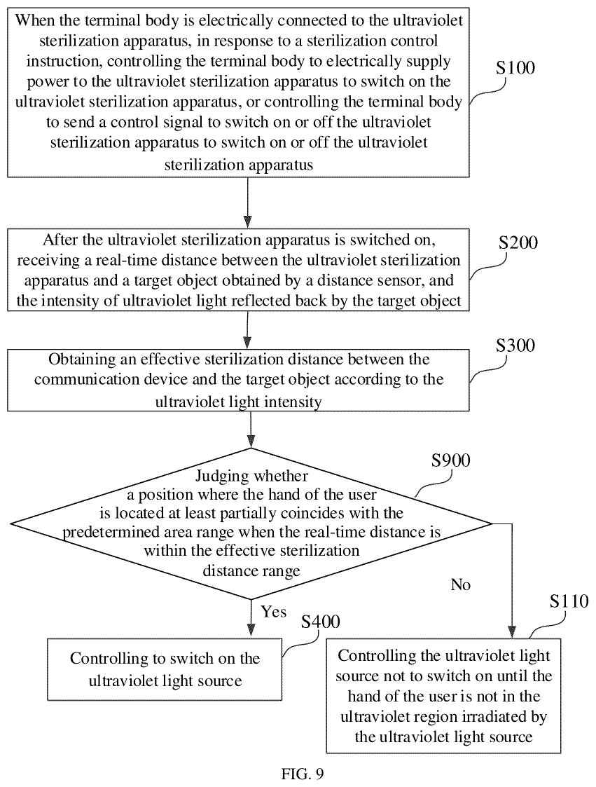

FIG. 9 is a schematic flow chart of a disinfection and sterilization method according to yet another specific embodiment of the present disclosure. As a specific embodiment of the present disclosure, before the ultraviolet light source 22 is controlled to be switched on, the embodiment further includes the steps below:
step S900, it is determined whether a position where the hand of the user is located at least partially coincides with the predetermined area range;
step S110, the ultraviolet light source is controlled not to switch on until the hand of the user is not in the ultraviolet region irradiated by the ultraviolet light source 22 when the position of the hand of the user at least partially coincides with the predetermined area range.

In the present embodiment, since the ultraviolet light is harmful to a person, it is necessary to determine whether the ultraviolet light source 22 irradiates a part of the human body before switching on the ultraviolet light source 22. Only when the hand of the user is not in the ultraviolet irradiation area, the ultraviolet light source 22 can be smoothly switched on for sterilization, thereby ensuring safety.

Thus, those skilled in the art will recognize that while various exemplary embodiments of the disclosure have been shown and described in detail herein, many other variations or modifications may be determined or derived directly in accordance with the present disclosure without departing from the spirit and scope of the disclosure. Accordingly, the scope of the present disclosure should be understood and interpreted to cover all such other variations or modifications.

What is claimed is:

1. A communication device, comprising a terminal body and an ultraviolet sterilization apparatus, wherein the terminal body is detachably and electrically connected to the ultraviolet sterilization apparatus, and the terminal body is used for providing electric energy for the ultraviolet sterilization apparatus; or
the terminal body is in wireless communication connection with the ultraviolet sterilization apparatus, and the terminal body is used for sending, to the ultraviolet sterilization apparatus, a control signal for switching on or switching off the ultraviolet sterilization apparatus.

2. The communication device according to claim 1, wherein
the ultraviolet sterilization apparatus comprises a housing and an ultraviolet light source provided at the housing, the housing is detachably connected to the terminal body.

3. The communication device according to claim 2, wherein
the housing is detachably attached to a surface of the terminal body or detachably sleeved on a rear side of the terminal body.

4. The communication device according to claim 3, wherein
the ultraviolet light source forms an image of a preset shape from at least one LED lamp bead, and the ultraviolet light source is located at a predetermined area range of the housing.

5. The communication device according to claim 4, wherein
the preset shape is a strip shape or a surface shape.

6. The communication device according to claim 4,
further comprising a distance sensor configured for detecting a distance between the ultraviolet sterilization apparatus and a target object;
wherein the terminal body further comprises a sterilization control unit; and the sterilization control unit is connected to the distance sensor, and is configured for sending a prompt message prompting the user to approach or move away from the target object according to the detection result of the distance sensor.

7. The communication device according to claim 6, wherein
the distance sensor is provided at the ultraviolet sterilization apparatus at a side of the ultraviolet light source.

8. The communication device according to claim 7, wherein
the sterilization control unit is further configured for obtaining an effective sterilization distance of the target object according to the received intensity of the ultraviolet light wave emitted by the target object, determining whether the target object is within the effective sterilization distance according to the distance obtained by the distance sensor and the effective sterilization distance, and sending a prompt message prompting the user to approach or move away from the target object when the target object is not within the effective sterilization distance.

9. The communication device according to claim 8, wherein
the sterilization control unit is further configured for calculating a distance between the target object and the ultraviolet light source based on a time of the ultraviolet wave emitted from the ultraviolet light source, a time of receiving the ultraviolet wave, and a wavelength of the ultraviolet wave after the distance sensor receives the ultraviolet wave emitted from the ultraviolet light source reflected from the target object.

10. The communication device according to claim 8, wherein
the distance sensor comprises a laser assembly, a receiving assembly and a processing module, when the distance sensor is switched on, the laser assembly emits laser light; after the receiving assembly receives the light wave reflected by the target object, the distance between the target object and the ultraviolet light source is calculated by the processing module.

11. The communication device according to claim 6, wherein
the terminal body further comprises a display unit, the display unit is connected to the sterilization control unit, and is configured for displaying the distance between the target object and the ultraviolet sterilization apparatus and the prompt message.

12. The communication device according to claim 11, wherein
the terminal body further comprises a rear camera for photographing an image at the rear of the terminal body; the rear camera is electrically connected to the sterilization control unit so as to transmit the photographed image information to the sterilization control unit; and the sterilization control unit then transmits the image information to the display unit and controls the display unit to display the image.

13. The communication device according to claim 12, wherein
a first opening is provided at the housing; and
when the terminal body is engaged with the housing, the rear surface of the terminal body abuts against the housing, and the rear camera is located at the first opening.

14. The communication device according to claim 6, wherein
the terminal body further comprises a laser control unit electrically connected with the laser assembly, so that the laser control unit controls the terminal body to supply power to the laser light source or to switch on or off the laser light source when receiving a laser control instruction.

15. The communication device according to claim 6, wherein
a laser light source is provided at the housing; and
the terminal body further comprises a laser control unit electrically connected with the laser light source, so that the laser control unit controls the terminal body to supply power to the laser light source or to switch on or off the laser light source when receiving a laser control instruction.

16. The communication device according to claim 15, wherein
the laser light source is provided on a side of the ultraviolet light source.

17. The communication device according to claim 6, wherein
a pressure sensor is provided at the left and/or right side of the housing or the terminal body; the pressure sensor is electrically connected to the sterilization control unit so as to transmit position information about a hand of the user holding the housing or the terminal body to the sterilization control unit after the pressure sensor monitors the position information; and the sterilization control unit determines, according to the position information, that the position of the hand of the user at least partially coincides with the predetermined area range, and controls whether to switch on the ultraviolet light source.

18. The communication device according to claim 17, wherein
when the pressure sensor is provided at the terminal body, second openings are correspondingly provided on both sides of the housing, so that when the pressure sensor is fitted or sleeved together with the terminal body, the pressure sensor is exposed to the outside.

19. A disinfection and sterilization method applied to the communication device according to claim 1, comprising:
when the terminal body is electrically connected to the ultraviolet sterilization apparatus, in response to a sterilization control instruction, controlling the terminal body to electrically supply power to the ultraviolet sterilization apparatus to switch on the ultraviolet sterilization apparatus, or controlling the terminal body to send a control signal to switch on or off the ultraviolet sterilization apparatus to switch on or off the ultraviolet sterilization apparatus.

20. The disinfection and sterilization method according to claim 19, comprising:
after the ultraviolet sterilization apparatus is switched on, receiving a real-time distance between the ultraviolet sterilization apparatus and a target object obtained by a distance sensor, and the intensity of ultraviolet light reflected back by the target object;
obtaining an effective sterilization distance between the communication device and the target object according to the ultraviolet light intensity;
controlling the switching on of the ultraviolet light source when the real time distance is within the effective sterilization distance range; and
when the real-time distance is not within the effective sterilization distance range, controlling the ultraviolet light source to be switched off, and sending a prompt message prompting the user to change the implementation distance.
